(19) United States
(12) Patent Application Publication
(10) Pub. No.: US 2015/0126957 A1
(43) Pub. Date: May 7, 2015

(54) ELONGATED PLEDGET FOR MEDICAL USE

(71) Applicants: François Marchal, Sarras (FR); Denis Artus, Parentis En Born (FR); Thierry Bouet, Annonay (FR)

(72) Inventors: François Marchal, Sarras (FR); Denis Artus, Parentis En Born (FR); Thierry Bouet, Annonay (FR)

(73) Assignee: LABORATOIRE TETRA MEDICAL, Annonay (FR)

(21) Appl. No.: 14/394,325

(22) PCT Filed: Apr. 12, 2013
(86) PCT No.: PCT/FR2013/050804

§ 371 Date: Dec. 9, 2014

(30) Foreign Application Priority Data
Apr. 13, 2012 (FR) 1201116

(51) Int. Cl. A61F 13/36

(52) U.S. Cl. CPC A61F 13/36
USPC 604/385.201

(57) ABSTRACT

The absorbent elongated pledget (1) includes two opposite width ends, the pledget being made from a flexible, hydrophilic material, and having a portion that can be detected from outside the body (3). The pledget is characterized in that at least one of the two width ends of same is folded on itself, the fold of the width end being held by a fastening element (2) in a fold forming a cylindrical shape of the pledget and allowing insertion into and removal from a trocar.

The present invention relates to an absorbent elongated device intended for minimally invasive surgery operations, using trocars.

In most of conventional (open) surgical operations or through endoscopic access, the operators need to stop bleedings.

For that purpose, they can use clips, electrocoagulation instruments, irrigation/suction devices, or finally surgical pledgets.

Pledgets are the device of choice because they are conformable, they have a high capacity of absorption relative to their weight and their surface, and they can be left in place during a time to separate anatomical structures.

In the specific case of the numerous operations using trocars, a conventional, folded pledget is not adapted because it is very difficult to insert it in the trocar. The operator further takes the risk to tear the device.

Similar objectives have been pursued by different inventions that propose different embodiments.

The patent of R. Seminara US 2007/0049860 describes a sponge. It differs from the present invention in that the selected material is uniquely expandable foam mounted on a handle. The invention is not exclusively intended for endosurgery. The use is different from a pledget. The object is not fully inserted inside the body.

The patent of Microtech WO 2005/053589 describes an haemostatic tubular material, of the PVA foam type. The latter is of single-shape absorbent plug nature. The present invention differs from it by the fact a portion of absorbent material may open out as an absorbent pledget. The described device is associated to a specific instrument of insertion. Moreover, the recovery method requires an adapted instrument end, but also a certain training to the gesture.

The patent of I. YOON WO 92/01433 describes a device designed for endoscopic surgery, based on an instrument. But the consumable is necessarily adapted to a single instrument. It allows absorption, irrigation and suction, but, as other inventions, is not intended to be fully inserted in the inner cavity. The absorbent material always remains attached to the trocar.

The patent U.S. Pat. No. 5,522,795 also describes an absorbent device for endoscopic surgery. This invention, a little like that of the above-mentioned YOON's one, is made of “a flexible material”, a small-size pledget, hence a low capacity of absorption, intimately linked to the inserting device. At no moment, the small absorbent pad fixed to its tubular portion is not intended to be detached and recovered, unlike the present invention.

Finally, another patent close to the present application, Shlain LEONARD, dated from 1992, i.e. one year before the USSC patent, also describes a flexible absorbent ribbon exiting from an inserting guide. This material remains attached to the instrument and is dependent on a specific inserting device.

It is also known by the document WO2004/110322 a pledget that is folded over itself and sewn along its four sides and that is therefore rectangular in shape, as shown in its FIG. 7 of trocar passage test. A radio-opaque wire is also fixed to the pledget.

The object of the patent is hence a device combining the characteristics of a pledget and a shape adapted to the easy insertion and removal through the surgical trocar lumen. The device is made of a flexible fibrous or spongy material, which is rolled or folded over, such terms being herein considered as equivalent within the context of the invention, in one dimension (in this case, a width end), to form a cylinder of diameter lower than the inner diameter of the usual trocars and able to partly unroll or unfold once out of the trocar to exhibit an increased contact surface. More particularly, the pledget, which is rectangular in shape, with two width ends and two length sides, is rolled or folded over one (or both) of its two width ends and this roll or fold is fastened by a fastening means that hence concerns only said width end. Therefore, the remaining of the pledget remains free to open out.

More precisely, the invention relates to an absorbent elongated pledget having two opposite width ends, said pledget being made from a flexible, hydrophilic material, and having a portion that can be detected from outside the body.

According to the invention, the pledget has at least one of its two width ends that is folded over itself, said fold of said width end being held by a fastening means in a fold forming a cylindroid shape of the pledget and allowing the insertion into and removal from a trocar.

In various embodiments of the invention, the following means are used, which can be used in isolation or according to any technically possible combination:
the pledget uses a non-deformable fabric in the greatest length of the object, and having a speed of absorption equivalent to that of the surgical pledgets,
the pledget is manufactured from a very absorbent and liquid retaining, biocompatible and sterilisable material,
the fastening means for holding the fold of the width end over itself is chosen among: a seam, a press-fit plastic sleeve, a High-Frequency or ultrasonic weld, or a bond or a hard wound adhesive,
the pledget is provided with a flexible fastener,
the flexible fastener is of variable length,
the flexible fastener is elastic,
the flexible fastener is fastened by the fastening means at the width end folded over itself,
the unfolded surface of absorbent material, i.e. that which is not fastened by the fastening means, is defined so that the fold forms a cylindrical shape of dimension compatible with the trocars usually used in surgery,
the absorbent and flexible material has uncrumbable and unfrayable, reinforced, welded or hemmed edges preventing the salting out of particles,
the folded-over material has a reduced surface, and the opening out of the portion of material outside the width end folded over itself and held by the fastening means has a significant surface allowing to form a barrier on the operation site.

The present invention will now be exemplified, without being limited thereby, by the following description of embodiments and modes of implementation, in relation with:

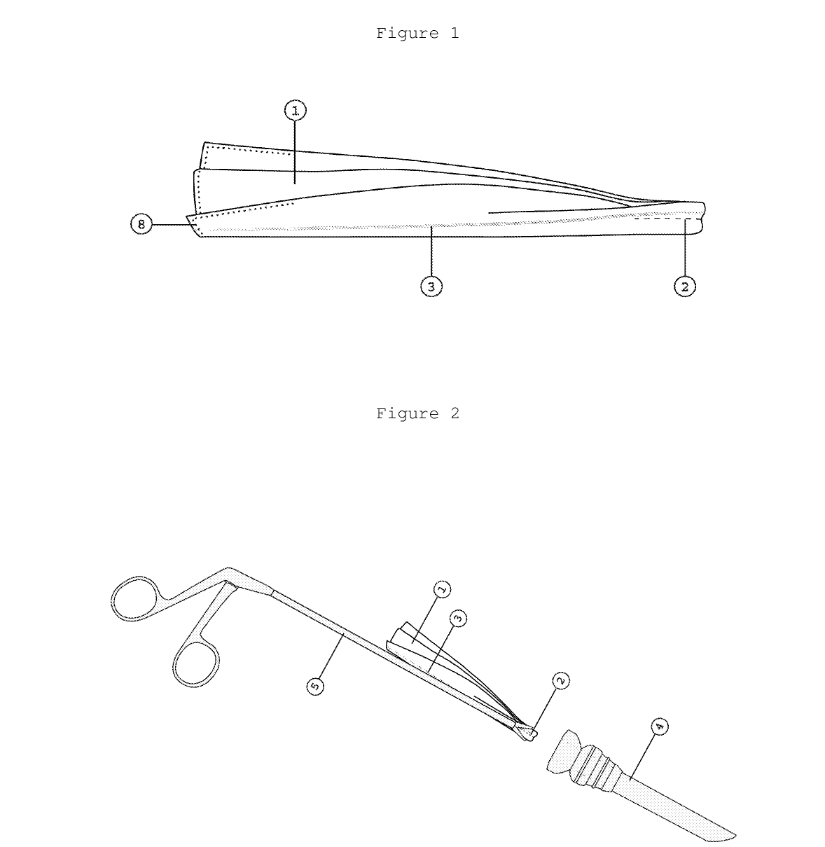

FIG. 1 that shows the pledget, one width end of which, which is folded over and fastened by a fastening means, has been sewn, providing it with a cylindroid shape or, more precisely, a conical/fan shape as shown, its non-sewn portion being free to open out,

FIG. 2 that shows the pledget gripped at its sewn width end to be inserted into a trocar,

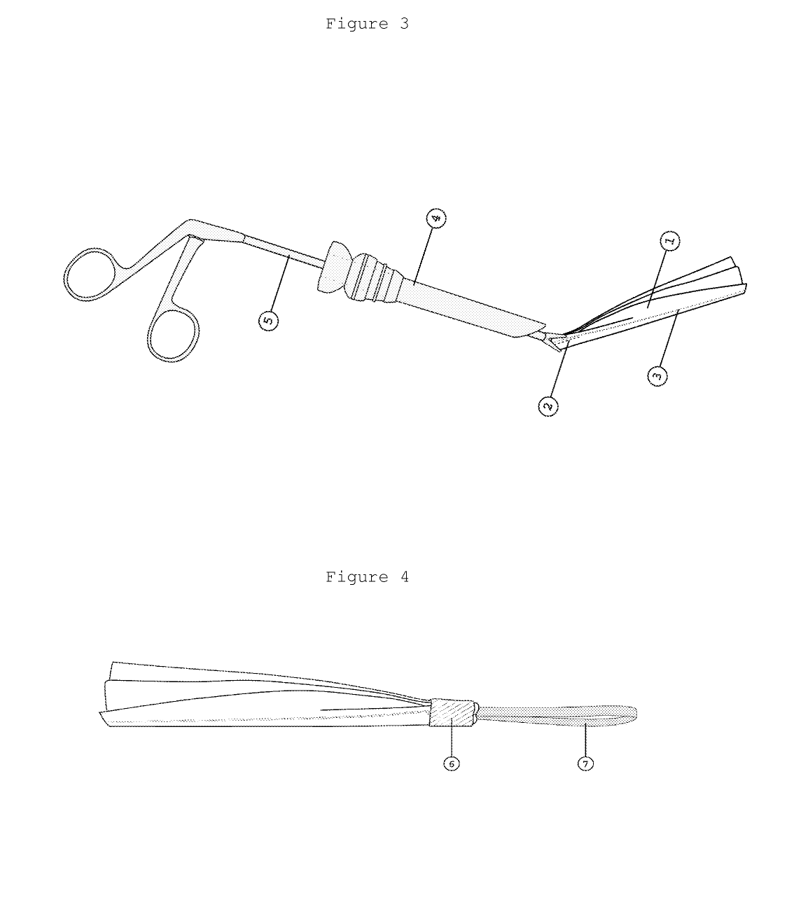

FIG. 3 that shows the pledget after being used, to be removed through the trocar, and

FIG. 4 that shows an alternative embodiment of the pledget, including a fastener.

A preferred embodiment is obtaining a flexible elongated body, and which is deformable as shown in FIG. 1. The deformable characteristic allows to grip the device with an endosurgery clamp 5 to insert and pull it into the trocar, as shown in FIG. 2, such that the absorbent body and the clamp jaws have together a size lower than the trocar lumen.

For absorption, most of the hydrophilic and flexible material may suit. They must have a great capacity of absorption in mass of absorbed water relative to the mass of the dry pledget, by way of example at least 8 grams per gram. The material may be a foamed polymer (polyurethane foam type), a thick non-woven (polyester, viscose or other hydrophilic polymer felt type) or a warp/weft fabric or a knit.

A preferential embodiment uses a flexible and resistant non-woven, also having a significant unfolded surface. The material is liquid retainer and sterilisable.

The strength and the deformation of the chosen material may be different according to the pulling direction. Even if the extraction by the trocar is made easy by the adapted shape of the pledget, the latter has to exhibit a minimum strength of 30 Newtons per cm and a low deformation (<15% of elongation at rupture). The pledget has to be non-deformable in the greatest length of the object.

The mode of link/fastening of the folded-over material at at least one of its two width ends may be a seam, a high-frequency or ultrasonic weld, a bound or the application of a press-fit plastic sleeve, or finally the application of a tightened rolled adhesive as shown in FIG. 4. Any other means for fastening the absorbent material to itself, for example a staple, is also possible. The folded-over material has a reduced surface, and the opening out of the non-sewn portion as unfolded has a significant surface allowing to form a barrier on the operation site. The fact that a portion of absorbent material can unfold as shown in FIG. 1 is important to be able, once open out, to constitute a temporary barrier. In other words, the fastening by the fastening means, seam or other, is not made over the whole length of the pledget nor over the whole periphery of the pledget but over only a folded-over edge of the pledget width, as shown. Therefore, the pledget, with its width end folded-over and fastened, exhibits a cylindroid shape that is similar to a cone or a fan. In a not-shown variant, each of the two width ends are folded over and fastened. The length of fastening in folded-over mode is determined by the desired flexibility for the whole. The chosen material has also to be biocompatible and compatible with a mode of sterilization to be used in an operating block.

The pledget has further a portion that can be detected from outside the body, which means, within the context of the invention, that the pledget can be detected by a radiology or ultrasound equipment, or even a metal detector, from outside the body.

The device may be continued by a textile or plastic fastener, which can be gripped by a clamp-type instrument, as shown in FIG. 4. Hence, the pledget may be provided with a flexible fastener 7 of variable length.

The selected material has also to exhibit a speed of absorption in relation with the need of the surgical gesture (for example, immersion time <10 seconds).

The surface of absorbent material is defined so that the fold forms a cylindrical shape of size compatible with the trocars usually used in surgery, by way of non-limitative example, of the order of 15 mm of diameter for the wider, down to 5 mm of diameter for the fine trocars.

The absorbent material must not exhibit risks of fraying or crumbling of particles or fibres on the edges, when impregnated of liquid or if it is gripped by the jaws of an instrument. In the case of a fibrous material, the edges on each side will be thermally treated (hot-melting or heat-setting). In the case of a woven or knitted material, a seam of the roundseam or hem type will be able to reinforce the object. An embodiment may be welded, reinforced, hemmed edges, preventing the salting out of particles as schematized in 8 in FIG. 1.

Finally, the invention of the present document has the advantage to use existing endoscopic surgery instruments, and does not need a specific training for being used.

At the extraction of the pledget, FIG. 3, from the endosurgical cavity, if the gripping of the pledget 1 by the clamp 5 is not in the axis of the trocar 3, the flexibility of the material nevertheless allows the proximal end of the pledget to be adapted to the trocar lumen. The pledget is the most often inserted in a dry state and removed in a damp state, hence a size smaller than the inner diameter of the trocar will favour the retaining of absorbed liquid in the operation site.

1. An absorbent elongated pledget (1) having two opposite width ends, said pledget being made from a flexible, hydrophilic material, and having a portion that can be detected from outside the body (3), characterized in that it has at least one of its two width ends that is folded over itself, said fold of said width end being held by a fastening means (2) in a fold forming a cylindroid shape of the pledget and allowing the insertion into and removal from a trocar.

2. The pledget according to claim 1, characterized in that it uses a non-deformable fabric in the greatest length of the object, and having a speed of absorption equivalent to that of the surgical pledgets.

3. The pledget according to claim 1, characterized in that it is manufactured from a very absorbent and liquid retaining, biocompatible and sterilisable material.

4. The pledget according to claim 1, characterized in that the fastening means (2) for holding the fold of the width end over itself is chosen among: a seam, a press-fit plastic sleeve, a High-Frequency or ultrasonic weld, or a bond or a hard wound adhesive (6).

5. The pledget according to claim 1, characterized in that it is provided with a flexible fastener.

6. The pledget according to claim 5, characterized in that the flexible fastener is of variable length (7).

7. The pledget according to claim 5, characterized in that the flexible fastener is fastened by the fastening means (2) at the width end folded over itself.

8. The pledget according to claim 1, characterized in that the unfolded surface of absorbent material is defined so that the fold forms a cylindrical shape of dimension compatible with the trocars usually used in surgery.

9. The pledget according to claim 1, characterized in that the absorbent and flexible material has uncrumbable and unfrayable, reinforced, welded or hemmed edges preventing the salting out of particles (8).

10. The pledget according to claim 1, characterized in that the folded-over material has a reduced surface, and the opening out of the portion of material outside the width end folded over itself and held by the fastening means (2) has a significant surface allowing to form a barrier on the operation site.

11. The pledget according to claim 6, characterized in that the flexible fastener is fastened by the fastening means (2) at the width end folded over itself.
